(19) United States
(12) Patent Application Publication
(10) Pub. No.: US 2009/0186778 A1
(43) Pub. Date: Jul. 23, 2009

(54) Method for analysis of multiple regions of DNA in single cells of uncultured microorganisms

(76) Inventors: Ramunas Stepanauskas, Nobleboro, ME (US); Michael Sieracki, Boothbay Harbor, ME (US)

Correspondence Address: WOLF GREENFIELD & SACKS, P.C., 600 ATLANTIC AVENUE, BOSTON, MA, 02210-2206, US

(21) Appl. No.: 12/009,582

(22) Filed: Jan. 18, 2008

(51) Int. Cl. C40B 40/06; C12Q 1/68; C40B 50/00; C12Q 1/70

(52) U.S. Cl. 506/16; 435/6; 435/5; 506/23

(57) ABSTRACT

Described herein are methods for single cell sorting and DNA analysis which permit metabolic mapping of taxonomically diverse microbial cells. Methods described herein encompass procedures for single-cell separation of individual uncultured cells, such as aquatic microbial cells, by fluorescence-activated cell sorting (FACS), subsequent single cell whole genome amplification (WGA), and downstream analyses of multiple regions of DNA.

### GOVERNMENT INTEREST

This invention was made with Government support under National Science Foundation Award #EF-0633142. The Government has certain rights in the invention.

### FIELD OF THE INVENTION

The present invention relates to methods for performing DNA analysis in uncultured microbial cells.

### BACKGROUND OF THE INVENTION

The identification of predominant microbial taxa with specific metabolic capabilities remains one the biggest challenges in environmental microbiology, due to the limits of current metagenomic and cell culturing methods.

PCR- and direct cloning-based sequencing of environmental DNA extracts have revealed enormous, previously unknown phylogenetic and metabolic diversity of prokaryotes (1-9). Although yet-uncultured taxa are believed to comprise more than 99% of all prokaryotes, their metabolic capabilities and ecological functions remain enigmatic, largely due to methodological limitations. For example, PCR-based clone libraries are intrinsically limited to the analysis of one gene at a time, with no direct way of linking libraries of diverse genes. Large-scale environmental shotgun sequencing, although useful for finding novel genes, is prohibitively expensive and to date is limited to only partial genome assembly of the most numerically dominant taxa in complex marine microbial communities (5, 9). Genomic analyses of large environmental DNA inserts can lead to remarkable discoveries, such as proteorhodopsin genes in bacteria (2). However, large insert-based function assignment is limited to situations where the metabolic gene of interest is located near taxonomic markers (e.g. ribosomal genes).

Thus, currently available culture-independent research tools are poorly suited for identification of microorganisms with specific metabolic characteristics. This significantly limits the progress in such diverse fields as biogeochemistry, microbial ecology and evolution, and bioprospecting.

### SUMMARY OF THE INVENTION

Described herein are methods for single cell sorting and DNA analysis which permit metabolic mapping of taxonomically diverse cells. Methods described herein encompass procedures for single-cell separation of individual uncultured cells, such as aquatic microbial cells, by fluorescence-activated cell sorting (FACS), subsequent single cell whole genome amplification (WGA), and downstream analyses of multiple regions of DNA. Also described are single amplified genomes (SAGs) and methods for constructing a library of SAGs. SAGs and SAG libraries are analyzed for the occurrence of specific DNA sequences, such as metabolic genes and/or genes that serve as taxonomic/phylogenetic markers. The methods and libraries of this invention can be used for taxonomic analysis and metabolic mapping of microorganisms such as marine bacterioplankton.

Described herein are methods for analyzing multiple regions of DNA in an individual uncultured cell. The method is useful, for example, for metabolic mapping of taxonomically diverse marine bacterioplankton. In some embodiments, methods described herein comprise obtaining a sample; sorting the sample by FACS to isolate/remove an individual cell, thereby obtaining an individual uncultured cell; amplifying the genome of the individual uncultured cell, thereby producing an amplified genome; and analyzing the amplified genome for DNA or genes that are within, or are present within, the cell. In some embodiments, the individual uncultured cell is a microbial cell. In certain embodiments the cell is an aquatic microbial cell. In some embodiments, the methods involve analyzing at least one region of DNA in an individual uncultured microbial cell. In a specific embodiment, methods comprise obtaining a marine bacterioplankton sample; sorting the sample by FACS to isolate or remove an individual marine bacterioplankton cell, thereby obtaining an individual marine bacterioplankton cell; amplifying the genome of the individual uncultured marine bacterioplankton cell, thereby producing an amplified genome; and analyzing the amplified genome for DNA or genes that are within, or are present within, the uncultured marine bacterioplankton cell. In specific embodiments, the methods involve analyzing at least one region of DNA in an individual uncultured marine bacterioplankton cell.

In some embodiments, the genome of the individual uncultured cell, such as an individual uncultured aquatic microbial cell, is amplified through whole-genome multiple displacement amplification (MDA). In some embodiments, analyzing at least one region of DNA within the cell is performed by combining the amplified genome of the cell with primers that are designed to amplify specific regions of DNA in the amplified genome, under conditions that result in hybridization of the primers to at least one specific region of DNA, thereby determining the occurrence of the specific region of DNA in the amplified genome. Analyzing regions of DNA within the cell can be performed for example by genomic sequencing.

In some embodiments, the methods described herein for analyzing regions of DNA within a cell can be used for analyzing taxonomic or metabolic markers, such as genes of biogeochemical significance in an individual uncultured cell, such as an aquatic microbial cell. Also described herein are methods for metabolic mapping comprising obtaining a microbial sample; sorting the sample by FACS, thereby obtaining an individual uncultured microbial cell; amplifying the genome of the individual uncultured microbial cell, thereby producing an amplified genome; analyzing at least one region of DNA within the cell, and associating at least one region of DNA within the cell with metabolic activity. In some embodiments the individual uncultured microbial cell is an aquatic microbial cell. In some embodiments, the method of metabolic mapping is a method of identifying genes of biogeochemical significance.

Also described herein are SAGs and SAG libraries and methods for the creation of SAG libraries from individual uncultured cells. In some embodiments, methods of creating a SAG library comprise: obtaining a sample of uncultured cells; sorting the sample, thereby obtaining individual uncultured cells, and amplifying the genomes of the individual uncultured cells, thereby producing a collection of single amplified genomes (SAGs) from individual uncultured cells. In some embodiments, the individual uncultured cells are microbial cells such as aquatic microbial cells, including, for example, uncultured marine bacterioplankton cells.

A SAG library, such as a SAG library created by a method disclosed herein, can be used for identifying metabolic genes, such as genes of biogeochemical significance. In some embodiments, a SAG library, such as a SAG library created by a method disclosed herein, can be used for whole-genome sequencing of SAGs. In some embodiments the SAG library comprises marine bacterioplankton SAGs.

### BRIEF DESCRIPTION OF THE DRAWINGS

FIG. 1 depicts maximum likelihood phylogenetic trees of bacterial SSU rRNA genes and proteorhodopsins. The SSU rRNA tree includes SAGs from (62) and all Flavobacteriaceae isolates with completed or currently undergoing whole genome sequencing. The proteorhodopsin tree includes SAGs from (62), and the most closely related sequences in GenBank, based on a BLASTP search. GenBank accession numbers are provided for each clone. Nodes marked with circles have >70% neighbor joining bootstrap support. Names of SAGs from (62), are in black rectangles.

FIG. 2 depicts maximum likelihood phylogenetic trees of the PufM and NasA. Included are protein sequences obtained from 100-cell MDA reactions and most closely related sequences in GenBank, based on BLASTP searches. GenBank accession numbers are provided for each clone. Nodes with circles have >70% neighbor joining bootstrap support. Names of SAGs from (62), are in black rectangles.

FIG. 3 demonstrates gel electrophoresis of multiple displacement amplification (MDA) products obtained using Protocol C: molecular weight ladder (lanes A and H), no-drop controls (lanes B-C), single bacterioplankton cells (lanes D-E), and 100 bacterioplankton cells (lanes F-G). The 0.75% TAE agarose gel was loaded with 3 uL of high molecular weight ladder (Invitrogen, Carlsbad, Calif.) and 10× diluted products of MDA reactions and then electrophorated at 0.5 V/cm for 5 hours.

FIG. 4 demonstrates real-time monitoring of multiple displacement amplification reactions of (A) standards prepared from human genomic DNA and (B) flow-cytometrically sorted material. Insert in A shows the log-linear standard curve for DNA concentration.

FIG. 5 demonstrates T-RFLP profiles of bacterial SSU rRNA genes obtained by (A) single cell MDA-PCR, (B) 100-cell MDA-PCR, and (C) 1000-cell semi-nested PCR. Panel A is a composite of six single-cell profiles. Insets show discrimination of the peaks near 90 bp with an expanded scale. All profiles were obtained using 27F-FAM and 907R primers and HhaI restriction endonuclease.

TABLE 1
Phylogeny of bacterial SSU rRNA genes obtained from single amplified genomes.

| | | | | | | |
|---|---|---|---|---|---|---|
| | | | | | T-RFL | T-RFL |
| | Lysis | | | | bp | bp |
| SAG ID | protocol | Genus¹ | Closest isolate² | Closest sequence³ | HhaI | (HaeIII) |

Flavobacteria/Flavobacteriaceae

| | | | | | | |
|---|---|---|---|---|---|---|
| MS021-5C | A | Kordia, 26% | Flavobacterium sp. | clone NorSea37 | 90 | 283 |
| | | | 3034 | AM279169, 96% | | |
| | | | AM110988, 91% | | | |
| MS024-2A | B | Kordia, 36% | Flavobacterium sp. | clone NorSea43 | 94 | No cut |
| | | | 3034 | AM279191, 99% | | |
| | | | AM110988, 91% | | | |
| MS024-3C | B | Cellulophaga, 80 | Cellulophaga sp. | clone 1D10 | 96 | 32 |
| | | | CC12 | AY274838, 99% | | |
| | | | DQ356487, 93% | | | |
| MS024-1F | B | Tenacibaculum, | Sponge bacterium | clone WLB13-197 | 90 | 281 |
| | | 98% | Zo9 | DQ015841, 96% | | |
| | | | AY948376, 97% | | | |
| MS056-2A | C | Ulvibacter, 99% | Ulvibacter litoralis | clone PB1.23 | 94 | 284 |
| | | | AY243096, 95% | DQ071072, 99% | | |

Sphingobacteria/Saprospiraceae

| | | | | | | |
|---|---|---|---|---|---|---|
| MS190-1F | B | Heliscomenobacter, | Saprospiraceae | clone SanDiego3-A7 | 92 | 407 |
| | | 55% | bacterium MS-Wolf [symbol] H | DQ671753, 100% | | |
| | | | AJ786323, 88% | | | |

Alphaproteobacteria/Rhodobacteraceae

| | | | | | | |
|---|---|---|---|---|---|---|
| S056-3A | C | Sulfitobacter, | Roseobacter sp. | clone F3C24 | 55 | 32 |
| | | 98% | AY167254, 99% | AY794157, 100% | | |
| MS024-1C | B | Jannaschia, 60% | Ophiopholis aculear | clone EB080-L11F12 | 55 | 32 |
| | | | symbiont | AY627365, 100% | | |
| | | | U63548, 99% | | | |
| MS190-2A | B | Jannaschia, 55% | Ophiopholis aculear | clone EB080-L11F12 | 55 | 32 |
| | | | symbiont | AY627365, 100% | | |
| | | | U63548, 99% | | | |
| MS190-2F | B | Loktanella, 41% | Octadecabacter | Rhodobacteraceae | 55 | 32 |
| | | | orientus KOPRI | bact. 183 | | |
| | | | 13313 | AJ810844.1, 99% | | |
| | | | DQ167247, 97% | | | |

Gammaproteobacteria/Oceanospirillaceae

| | | | | | | |
|---|---|---|---|---|---|---|
| MS024-3A | B | Balneatrix, 24% | Marine | clone Ant4D3 | 575 | 413 |
| | | | gammaproteobacter [symbol] m | DQ295237, 99% | | |
| | | | HTCC2120 | | | |
| | | | AY386340, 90% | | | |

Gammaproteobacteria/Comamonadaceae

| | | | | | | |
|---|---|---|---|---|---|---|
| MS024-2C | B | Delftia, 100% | Delftia acidovorans | Delftia | 203 | 197 |
| | | | AM180725, 99% | acidovorans | | |
| | | | | AM180725, 99% | | |

¹Determined by RDP Classifier. Both type and non-type “good” ≧1,200 bp sequences were used. Numbers indicate confidence level for genus identification.
²Determined by RDP Seqmatch. Provided are sequence ID, accession number, and sequence identity to the SAG
³Determined by NCBI BLAST-N. Provided are sequence ID, accession number, and sequence identity to the SAG [symbol]
indicates data missing or illegible when filed

TABLE 2
PCR primers.

| Gene | Primers | Product, bp | References |
|---|---|---|---|
| Bacterial SSU rRNA | 27F, 519F, 907R, 1492R | various | (51, 52) |
| Archaeal SSU rRNA | S-D-Arch-0344-a-S-20, 907R | 550 | (53, 54) |
| Eukaryote SSU rRNA | EUK328f, EUK329r | 1500 | (55, 56) |
| Proteorhodopsin | o-PR2, o-PR3 | 330 | (37, 40) |
| Bacteriochlorophyll, pufM | pufM_228F, pufM_228R | 228 | (57) |
| Nitrogenase, nifH | nifUP, nifDN, NifH3, NifH4 | 450 | (27, 58) |
| Assimilatory nitrate reductase, nasA | nas22, Nas1933, nas964, | 771 | (28, 59) |
| | nasA1735 | | |

### DETAILED DESCRIPTION OF THE INVENTION

Aspects of the invention relate to the development of procedures for high-throughput single-cell separation from environmental samples by fluorescence-activated cell sorting (FACS), and subsequent single cell whole genome amplification (WGA). The methodology of the invention has multiple applications, including: availability of partial or whole genomes of yet-uncultured microorganisms, including rare species; genetic variation studies at the organismal rather than population level; and matching of taxonomy/phylogeny and metabolism in uncultured microorganisms. The latter is exemplified by the construction of single amplified genome (SAG) libraries, including a library of 11 SAGs constructed from Gulf of Maine bacterioplankton, and used for taxonomy/phylogeny and metabolism matching. An important advantage of this approach is its ability, compared with presently available approaches, such as metagenomics, to link taxonomic and metabolic markers in single uncultured microorganisms, even in those instances in which the taxonomic and metabolic markers are located far apart on a chromosome.

Described herein is DNA analysis in individual cells, rather than in environmental extracts containing pooled DNA from multiple organisms. The strategy of sequencing DNA from individual cells of environmental microorganisms has begun to gain momentum recently, with implementations of single cell multiplex PCR in termite gut microbiota by Ottesen et al. (10) and partial genome sequencing of single cells of Prochlorococcus by Zhang et al. (1). Described herein are methods involving single cell FACS and WGA, and the use of amplified genomes in downstream analysis of multiple loci.

Aspects of the invention relate to analysis of microbial cells. As used herein, the term microbial refers to “relating to microorganisms,” and the term “microorganism” refers to an organism of microscopic size. The term microbial cell includes all cells that are microorganisms. In some embodiments, the microbial cell is an aquatic microbial cell. The term “aquatic microbial cell” refers to a cell of a microorganism that is taken from an aqueous environment. In some embodiments, an aquatic microbial cell is a marine bacterioplankton. As used herein the term “plankton” refers to small, usually microscopic, organisms that float or weakly swim in salt or fresh water. “Bacterioplankton” refers to the bacterial and archael component of plankton. “Marine bacterioplankton” refers to bacterioplankton that are found in the ocean and/or whose natural habitat is the ocean. In some embodiments, a microbial cell may be obtained from an environmental soil sample.

Aspects of the invention relate to obtaining and sorting individual uncultured cells such as microbial cells. For example, individual microbial cells may be obtained from an environmental sample, such as a water column. Prior to sorting and selection of an individual cell, the sample may include a plurality of cell types, which can be homogeneous or heterogeneous. As used herein, the term “uncultured cell” refers to a cell that has not been adapted to grow in the laboratory. As used herein an “individual uncultured cell,” produced through cell sorting, refers to a cell that is substantially free of other cells and extra-cellular DNA. These can also be referred to, respectively, as “uncultured cell” and “individual uncultured cell.”

According to methods described herein, sorting a sample to obtain individual cells from the sample, is achieved through flow cytometry, using fluorescence-activated cell sorting (FACS). Compared to alternative methods, FACS offers several critical advantages, including high throughput rates and the ability for automated sorting of targeted cells, based on cell size and fluorescence signals of natural cell components and fluorochromes (13). Furthermore, cell separation by FACS creates microsamples containing the target cell and only 3-10 pL of sample around it (13). This reduces the co-deposition of extracellular DNA, which in marine waters occurs at concentrations similar to cell-bound DNA (14, 15). In some embodiments, a high-speed, droplet-based fluorescence-activated cell sorting (FACS) system is used. It should be appreciated that a variety of FACS systems are compatible with the instant invention, as would be familiar to one of ordinary skill in the art. In certain embodiments, a MoFlo (Dako Cytomation, Carpenteria, Calif.) flow cytometer equipped with the CyClone robotic arm is used.

In some embodiments, the sample is diluted and/or stained prior to FACS analysis. The sample can be stained with a nucleic acid stain. Some non-limiting examples of nucleic acid stains, available from Molecular Probes include: SYBR Green, SYBR Green II, SYTOX Green, SYTOX Blue, SYTOX Orange, POPO-1, BOBO-1, YOYO-1, TOTO-1, JOJO-1, POPO-3, LOLO-1, BOBO-3, YOYO-3, TOTO-3, PO-PRO-1, BO-PRO-1, YO-PRO-1, TO-PRO-1, JO-PRO-1, PO-PRO-3, LO-PRO-1, BO-PRO-3, YO-PRO-3, TO-PRO-3, TO-PRO-5, SYTO 40, SYTO 41, SYTO 42, SYTO 43, SYTO 44, SYTO 45, SYTO RNASelect, SYTO 9, SYTO 10, SYTO BC, SYTO 13, SYTO 16, SYTO 24, SYTO 21, SYTO 27, SYTO 26, SYTO 23, SYTO 12, SYTO 11, SYTO 20, SYTO 22, SYTO 15, SYTO 14, SYTO 25, SYTO 86, SYTO 81, SYTO 80, SYTO 82, SYTO 83, SYTO 84, SYTO 85, SYTO 64, SYTO 61, SYTO 17, SYTO 59, SYTO 62, SYTO 60, SYTO 63, Acridine homodimer, Acridine orange, 7-AAD (7-amino-actinomycin D), Actinomycin D, ACMA, 4,6-diamidino-2-phenylindole DAPI, Dihydroethidium, Ethidium bromide, Ethidium homodimer-1 (EthD-1), Ethidium homodimer-2 (EthD-2), Ethidium monoazide, Hexidium iodide, Hoechst 33258 (bis-benzimide), Hoechst 33342, Hoechst 34580, Hydroxystilbamidine, LDS 751, Nuclear yellow, Propidium iodide.

In some embodiments, aquatic samples, such as marine samples, are diluted in DNA-free solution and sorted in “purify 0.5 drop” mode, which minimizes the risk of delivery of more than one cell per droplet. The formation of stable cell aggregates that have fluorescence and light scatter properties similar to single cells are uncommon in marine samples and in the unlikely event of their occurrence, their presence would likely be detected by downstream molecular tools. Dividing cells, colonies, and other aggregates of genetically identical cells would not interfere with the methods of the invention.

Prevention of sample contamination with DNA represents a technical challenge of single cell selection and genome amplification, due to the requirements for single-cell analysis. Potential sources of DNA contamination include: extracellular DNA in field samples; flow cytometer sheath fluid; post-sort reagents; and post-sort workspace. Assuming about 1 ng/mL extracellular DNA (including viral) in surface ocean (15), one can estimate that the average amount of extracellular DNA delivered with each sort droplet using procedures of the instant invention, is equivalent to 100-1000 basepairs, or less than 1 gene. This level of contamination would not interfere with downstream genome assembly and would have a negligible probability of false positives in downstream PCR.

In some embodiments, in order to remove DNA contaminants from sheath fluid, a stringent procedure involving sheath fluid line replacement, multi-stage rinse, and in-house production of organics-free sheath fluid, is followed. In initial studies, no DNA could be detected in 1 uL aliquots of sheath fluid by quantitative real-time PCR employing Bacteria-specific 16S rRNA primers. Only about 1 nL sheath fluid accompanies each cell during sorting, demonstrating that the sheath fluid cleanup procedures of the instant invention are sufficient for single-cell analyses.

Some molecular biology reagents, particularly Taq polymerase, may contain significant amounts of microbial DNA (63). Appropriate blank treatments are used to verify that the cell lysis and WGA reagents are DNA-free. If necessary, DNA contaminants can be removed from problematic reagents using DNase (64, 65), ultrafiltration (66, 67), or UV irradiation (68, 69), depending on the properties of the reagent. The efficiency of these procedures has been tested in prior studies (70). Contamination from workspaces is prevented through sample handling in UV-treated enclosures, physical separation of pre- and post PCR processes, and workspace treatment with DNA-degrading agents, such as bleach and DNAZap (Ambion). To validate the efficiency of clean procedures, appropriate blanks are included in each experimental setup. In addition, to verify that genetic material from only one cell is amplified in positive treatments, the diversity of a marker gene such as the 16S rRNA gene is determined in each WGA reaction by terminal restriction fragment length polymorphism (T-RFLP).

### Whole Genome Amplification

According to methods described herein, following sample sorting to obtain an individual cell, the individual cell is lysed and then substantially the whole genome of the cell is amplified through whole genome amplification (WGA), producing a single amplified genome (SAG). Conventional DNA extraction procedures are not applicable for single cell analyses due to the substantial DNA loss during the process. Instead, according to the instant invention, WGA is performed directly on cell lysates. In some embodiments, the cells are sorted into plates such as 96-well PCR plates pre-loaded with cell lysis buffer. To verify sort precision, multiple blanks are included in each plate, to which cell-free sheath fluid droplets are deposited. The absence of DNA in the blanks can be verified by WGA followed by PCR of a marker gene such as bacterial and archaeal 16S rRNA. It should be appreciated that a variety of cell lysis conditions and protocols known to those of skill in the art can be used with the instant invention. In some embodiments, the cells are lysed through cycles of heating and cooling. In other embodiments, the cells are lysed through alkaline lysis on ice. In certain embodiments, cell lysis is performed according to a protocol accompanying a REPLI-g kit (Qiagen).

Following lysis of the cell, the genome of the cell is amplified through WGA. Traditional sequencing technologies require nanogram to microgram DNA templates and are not capable of direct sequencing of individual DNA molecules. Such techniques require DNA pre-amplification to sequence genes or genomes from individual cells. For the analysis of up to two loci per cell, single cell multiplex PCR has been used in medical research since the 1980's (16) and was recently employed in an environmental microbiology study (10). As a more versatile alternative, allowing for analysis of an unlimited number of loci, several methods have been suggested for whole genome amplification, including degenerated oligonucleotide primed PCR (DOP), primer extension preamplification (PEP), ligation-mediated PCR, and multiple displacement amplification (MDA) using phi29 or Bst DNA polymerases (17, 18). MDA, as would be familiar to one of skill in the art, refers to a method of WGA that uses random priming. It should be appreciated that a variety of techniques suitable for whole-genome amplification can be used with the instant invention. In certain embodiments, phi29-based MDA is employed.

In some embodiments, MDA is performed using a REPLI-g kit (Qiagen), and Phi29 polymerase. Phi29-based MDA is efficient for whole-genome amplification, with a low error and bias (17, 18), and is capable of generating micrograms of genomic DNA from nanogram-sized samples (19-21). Recently, Phi29-based MDA was used on single human (18, 22, 23), Escherichia coli (24), and Prochlorococcus (11, 25) cells. A virtually unlimited number of downstream PCR, targeting diverse regions of DNA, can be performed on Phi29 products.

Analysis of DNA within an Amplified Genome

In some embodiments of the method described herein, following amplification of the genome of a cell, at least one region of DNA from the genome of the cell is analyzed. In some embodiments, analysis of DNA is conducted by PCR amplification of specific regions of the genome, using specific primers. PCR analysis involves combining the genome of the cell, amplified through WGA, with primers designed to amplify specific regions of DNA in the amplified genome, under conditions familiar to one of ordinary skill in the art, that result in hybridization of the primers to at least one specific region of DNA. PCR analysis can be used to detect the occurrence of a specific region of DNA in an amplified genome, such as the occurrence of a certain gene or a family of genes. In some embodiments, detecting the occurrence of a gene involves determining the presence or absence of a gene. PCR analysis can also be conducted to detect a variant or mutated form of a gene or family of genes. In some embodiments, PCR amplification can be followed by analysis of amplification products on an analytical gel and/or by sequencing of the PCR-amplified products. In some embodiments, the specific region of DNA that is amplified through PCR is a protein coding region of DNA, while in other embodiments it is a region of DNA that is not protein coding.

In some embodiments, genomic sequencing can be performed on the amplified genome, with or without a PCR amplification step, using specific primers, to detect specific regions of DNA. In some embodiments, the partial or whole genome of the individual uncultured cell will be sequenced after genome amplification. In other embodiments, only certain regions of the genome of the individual uncultured cell will be sequenced following genome amplification. In some embodiments, Southern, Northern, or Western blots can be used to detect specific genes or gene families, or the gene products (RNA or protein) produced by specific genes or gene families, in an amplified genome. In other embodiments, DNA microarrays or protein arrays can be used to detect specific regions of DNA, such as genes or gene families, or proteins produced by genes and gene families in an amplified genome.

In some embodiments, after cell lysis and WGA, in order to verify that samples were amplified from single cells, homogeneity of the 16S rRNA gene amplicons is verified by T-RFLP analysis using fluorescently labeled primers specific for Bacteria (52) and Archea (40). Only WGA products generating a single 16S rRNA gene T-RFLP peak are selected for further analyses. In some embodiments, an additional quality control measure consists of PCR-screening, sequencing, and matching genes such as 16S rRNA for phylogenetic/taxonomic analyses, and recA, a ubiquitously distributed and conserved protein-encoding gene for which a substantial database is available (71).

In some embodiments, the following criteria are used as guidelines in determining which WGA products are chosen for further analyses: Phylogenetically interesting microorganisms, such as yet uncultured phylotypes that are rarely encountered in metagenomic libraries, and whose genome sequence data would be particularly difficult to obtain using methods other than single cell WGA; and phylotypes for which novel functions were indicated by the analysis of metabolic genes. In some embodiments, if WGA products meeting these criteria are found, these amplified genomes can be candidates for whole genome or partial genome sequencing and annotation.

### Matching Taxonomy and Metabolism

The method described herein has the ability to detect multiple regions of DNA, such as multiple genes, in individual uncultured cells, such as cells of uncultured microorganisms, even in those cases in which the genes are located far apart on the chromosome. In some embodiments, analysis of a specific region of DNA, such as specific genes or gene families, in an individual cell is used for taxonomic analysis. As used herein, “taxonomic analysis” refers to detection or identification of taxonomic markers. As used herein a “marker” refers to a specific region of DNA that is detectable or identifiable. As used herein a “taxonomic marker” refers to a marker that can aid in the naming of an organism and/or the assignment of an organism to a taxa. A taxonomic marker can also be used for investigating phylogeny. As used herein, “phylogeny” refers to the evolutionary history of a taxonomic group. Thus a taxonomic marker may also serve as a phylogenetic marker. In some embodiments, the taxonomic markers are ribosomal RNA genes, such as the 16S, 18S, 23S and 28S rRNA genes. In some embodiments, analysis of a taxonomic marker refers to detection of the occurrence of the marker, such as the presence or absence of a marker. In other embodiments, analysis of a taxonomic marker refers to detection of a specific variant or form of the marker in a cell.

In some embodiments, analysis of a specific region of DNA, such as specific genes or gene families, in an individual cell, is used to detect metabolic markers. Metabolic activity refers to any activity that pertains to metabolism. The term “metabolism” encompasses all of the biochemical reactions that take place in a cell or organism. As used herein, “metabolic marker” refers to any marker that could be associated with cellular metabolism. As used herein, the term “metabolic mapping” refers to analysis of regions, typically specific regions, of DNA in a cell and the association of a specific region(s) of DNA in a cell with a metabolic function.

Cellular metabolism can produce energy for a cell. Thus identifying metabolic markers in microorganisms through metabolic mapping, provides a valuable tool for biogeochemical research through the identification of genes of biogeochemical significance. As used herein, “a gene of biogeochemical significance” refers to any gene that is associated with biologically mediated reactions that are significant for global energy or elemental transformation. In some embodiments, genes of biogeochemical significance include: bacteriochlorophyll pufM (72), proteorhodopsin (40), nitrogenase, and assimilatory nitrate reductase nasA (73). Bacterial proteorhodopsins (2) and bacteriochlorophylls (26) are photometabolic systems, recently recognized for their ubiquity and likely significance in the global carbon and energy fluxes. Nitrogenase is a key enzyme in the fixation of N₂, effectively controlling primary production in vast areas of the ocean and appears to be possessed by some heterotrophic bacterioplankton (27). Assimilatory nitrate reductase enables some heterotrophic bacteria to use nitrate and in this way compete with phytoplankton for the upwelled nitrogen (28). So far little is known about the taxonomic composition of microorganisms carrying these genes in marine environments. It should be appreciated that the above-mentioned genes are non-limiting examples of genes of biogeochemical significance and that any gene that is associated with metabolism may be compatible with the instant invention. In some embodiments, taxonomic identification (for example using 16S rDNA or recA) and protein-encoding gene verification is performed based on GenBank BLAST (47) and Ribosomal Database Project Classifier and Seqmatch search tools (48). In some embodiments, metabolic mapping may be a method for taxonomic analysis. In some embodiments, taxonomic analysis and/or metabolic mapping is used to identify genes of biogeochemical significance.

In some embodiments, following WGA of a single cell, the whole genome or partial genome of the cell is sequenced. With whole- or partial-genome sequence information, sequence-based annotation, for example using publicly available databases, can then be performed. As used herein the term “annotation” refers to assigning a predicted biological function to a gene. Sequence-based annotation refers to assigning a predicted biological function to a gene based on its DNA sequence. Several factors are relied upon in providing annotation for new sequences, including comparison to sequences in other species and homology calculations, identification of protein domains, and prediction of protein-protein interactions. These factors assist in determining an initial predicted annotation of a gene and an accompanying predicted function.

In some embodiments of the invention, genome sequence data of a bacterium is used for the reconstruction of its metabolic network (74). Sequence-based annotation of the genome of the bacterium, using databases such as GenBank BLAST or FASTA, provides assignment of molecular function to genes encoded for by the genome of the bacterium. Following this step, annotated genes can be searched on databases that provide information on metabolic pathways and reactions. Some non-limiting examples of resources that can be used for reconstructing the metabolism of an organism based on its genomic sequence include KEGG (75) and MetaCyc (76). Further resources for metabolic reconstruction can be found in (74), which is hereby incorporated by reference. It should be appreciated that sequence based annotation and its use in metabolic mapping serves as a guideline, which can then be verified experimentally through functional analysis.

In some embodiments of the invention, whole genome sequencing is not necessary for downstream analysis of genes or metabolic mapping of the organism. With an amplified genome, specific genes that have been annotated in other organisms can be targeted for analysis in the genome of the organism under study, for example by PCR amplification and/or sequencing. In some embodiments, the presence or absence of at least one region of DNA or the presence or absence of a variant of at least one region of DNA will be sufficient for taxonomic analyses or metabolic mapping. In other embodiments taxonomic analyses or metabolic mapping may involve the sequencing and identification of many regions of DNA from the amplified genome of a single cell.

In some embodiments, associating at least one region of DNA within the amplified genome of a cell, with metabolic activity, involves designing primers that will hybridize to a region of DNA that has been previously linked to a metabolic activity, and identifying the occurrence of this region of DNA in the amplified genome of the cell. In other embodiments, associating at least one region of DNA within the amplified genome of a cell, with metabolic activity, involves sequencing the partial or full genome of the cell, using the sequence information generated by this approach for annotation, and then identifying through homology, or through reconstruction of a metabolic network, regions of DNA that have been linked to metabolic function.

### SAG Libraries

Aspects of the invention relate to single amplified genomes (SAGs), and the creation of SAG libraries. As used herein the term “single amplified genome” or “SAG” refers to the amplified genome of a single cell. It should be appreciated that a SAG can be produced from any single cell from any organism. A “SAG library” refers to a collection of one or more single amplified genomes. According to methods of the invention, a SAG library is created through sorting a sample into individual uncultured cells, and amplifying the genomes of the single cells to produce a collection of amplified genomes from single cells. In some embodiments the cells are microbial cells such as aquatic microbial cells.

Similar to the analysis described for the amplified genome of a single cell, a SAG library of genomes, amplified from single cells, can be analyzed for the presence and DNA sequences of specific regions of DNA representing taxonomic and/or metabolic markers. Thus, a SAG library can be used for identifying metabolic markers such as genes of biogeochemical significance. In some embodiments SAG libraries are used for whole genome sequencing of SAGs.

As described further in the Examples, a library of 11 SAGs was constructed from Gulf of Maine bacterioplankton (62). The SAG library genomes were analyzed for the presence and DNA sequences of genes representing phylogenetic or taxonomic markers (SSU rRNA) and several significant biogeochemical functions in marine ecosystems (proteorhodopsin, bacteriochlorophyll, nitrogenase, and assimilatory nitrate reductase). The library consisted of five flavobacteria, one sphingobacterium, four alphaproteobacteria, and one gammaproteobacterium. Most of the SAGs, apart from alphaproteobacteria, were phylogenetically distant from existing isolates, with 88-97% identity in the 16S rRNA gene sequence. Thus, single cell MDA provided access to the genomic material of numerically dominant but yet-uncultured taxonomic groups. Two out of five flavobacteria in the SAG library contained proteorhodopsin genes, suggesting that flavobacteria are among the major carriers of this novel photometabolic system. The pufM and nasA genes were detected in some 100-cell MDA products but not in SAGs, demonstrating that organisms containing bacteriochlorophyll and assimilative nitrate reductase constituted <1% of the sampled bacterioplankton. Thus SAGs and SAG libraries provide a valuable research tool for taxonomic analysis and metabolic mapping.

The teachings of all references cited herein are hereby incorporated by reference in their entirety.

The present invention is illustrated by the following examples, which are not intended to be limiting in any way.

### EXAMPLES

### Example 1

### Taxonomic Composition of Single Amplified Genome (SAG) Library

The SSU rRNA gene was successfully PCR-amplified and sequenced from 12 out of 48 single cell MDA reactions (Table 1). The SAG MS024-2C was identified as a contaminant and excluded from further analyses. The remaining SAG library consisted of five flavobacteria, one sphingobacterium, four alphaproteobacteria of the Roseobacter lineage, and one gammaproteobacterium, all most closely related to marine isolates and clones. Diverse representatives of the Roseobacter lineage are readily isolated and are relatively well studied (29, 30). Accordingly, SSU rRNA genes of the four alphaproteobacterial SAGs were 99% identical to existing isolates. In contrast, all flavobacterial, sphingobacterial and gammaproteobacterial SAGs were phylogenetically distant from established cultures, with 88-97% identities in the SSU rRNA gene. Flavobacteria as a group are proficient degraders of complex biopolymers, including cellulose, chitin, and pectin (31). Thus, certain Flavobacteria taxa may play important and specialized roles in microbial food webs and may be attractive for bioprospecting. Single cell MDA provided access to the unique genomic material of these yet-uncultured taxa at individual organism level. In difference to the single cell multiplex PCR (10, 16), which enables analysis of up to two loci per cell, our approach generated a large quantity of high molecular weight whole genome amplification products. This material can be used in a virtually unlimited number of downstream PCRs (see below) and hybridization analyses and may be suitable for genomic sequencing (11).

Only high nucleic acid bacterioplankton was analyzed in this study, which comprised 57% of all heterotrophic prokaryotes in the sample. This may have biased the taxonomic composition of the SAG library, likely explaining the lack of SAR11 and some other ubiquitous groups (32). Nevertheless, the predominance of Bacteroidetes in the SAG library was unexpected. Alphaproteobacteria typically dominate SSU rRNA PCR clone libraries of marine surface bacterioplankton, while Bacteroidetes constitute <3% of all marine clones (33). In contrast, studies employing fluorescent in situ hybridization (34, 35), quantitative PCR (36) and metagenomics (5, 8, 9) suggest a higher proportion of Bacteroidetes (particularly Flavobacteria), in some cases >70% of the total bacterioplankton (31). This contradiction may be caused by PCR and/or cloning biases against Flavobacteria (31). Interestingly, the ratio of Alphaproteobacteria versus Bacteroidetes in our SAG library was 0.7 (Table 1), while the corresponding ratio of community T-RFLP peak areas at 55 bp (assumed Roseobacter lineage) and at 90-96 bp (assumed Bacteroidetes) was 1.0 (100 cell MDA-PCR) and 3.5 (1000 cell semi-nested PCR) (FIG. 5). This discrepancy is supportive of a PCR bias against Bacteroidetes, which would affect T-RFLP profiles, especially those based on two rounds of semi-nested PCR. On the other hand, the construction of SAG library was insensitive to PCR biases and does not involve cloning. Thus, scaled-up SAG libraries may become an ultimate tool for quantitative bacterioplankton analyses at high phylogenetic resolution. The advantage of SAG screening over fluorescent in situ hybridization, another taxon-specific quantification method, is demonstrated by the extraction of high-resolution phylogenetic information through sequencing of the entire SSU rRNA gene, as well as protein-encoding loci (see below).

Proteorhodopsin genes were detected by PCR and confirmed by sequence analysis in two out of eleven SAGs (FIG. 1). In addition, PCR-screening detected proteorhodopsin genes in all twelve 100-cell MDA reactions. Accordingly, Sabehi et al. (37) estimate that 13% bacterioplankton in the photic zone of the Mediterranean and Red seas carried proteorhodopsin genes. Our study provides further evidence that proteorhodopsin-containing microorganisms comprise a significant fraction of marine bacterioplankton.

Interestingly, both proteorhodopsin-positive SAGs were Flavobacteria, providing the first evidence that proteorhodopsins are common in the numerically abundant representatives of this taxonomic group (FIG. 1). The presence and photometabolic functionality of proteorhodopsins in Flavobacteria was recently confirmed by genome sequencing of four isolates (38). The first indication of proteorhodopsins in Flavobacteria was obtained from shotgun sequencing of Sargasso Sea microbes, where a proteorhodopsin gene was found on a scaffold also containing a DNA-directed RNA polymerase sigma subunit (rpoD) typical of Bacteroidetes (5). Bacterial proteorhodopsins were first discovered by screening environmental BAC libraries (2). Using this technique, several Gammaproteobacteria, Alphaproteobacteria and Euryarchaea were identified as proteorhodopsin hosts (39, 40). However, this approach has so far not indicated the presence of proteorhodopsins in Flavobacteria, possibly because the proteorhodopsin and SSU rRNA genes are too far apart. Studies based on community proteomics (41), community PCR (42, 43), community shotgun sequencing (5, 9) and PCR-screening of metagenomic BAC libraries (39) demonstrated high diversity of proteorhodopsins in the ocean, although the vast majority of their hosts remain unknown. So far only five marine isolates have been reported to contain proteorhodopsin genes, including alphaproteobacterium Pelagibacter ubique (44) and four Flavobacteria (38). Here we demonstrate how single cell MDA-PCR can provide a powerful and relatively inexpensive tool for the phylogenetic mapping of this biogeochemically important gene, independent of gene's position on the chromosome or host cultivability.

The two SAG proteorhodopsins were most closely related (up to 71% identity) to proteorhodopsins from four Flavobacteria isolates and to a group of environmental clones from the North Atlantic (FIG. 1). Consistent with the flavobacterial isolates and near-surface environmental sequences, both SAGs had methionine at amino acid position 105 (eBAC31A08 numbering), indicative of absorption maxima near 530 nm (green light) (38). In general, phylogenetic relationships among proteorhodopsins and SSU rRNA genes mirrored each other, providing no evidence for recent cross-taxa horizontal transfer events like those observed in Archaea (40). On the other hand, the presence of proteorhodopsin genes was inconsistent among some closely related Flavobacteria, e.g. Polaribacter filamentus 215 and P. irgensii 23-P (FIG. 1), suggesting recent proteorhodopsin gene losses. Interestingly, proteorhodopsin genes closely related to Flavobacterial SAGs and isolates were present among environmental clones from the North Atlantic, Mediterranean Sea, and Red Sea, indicating that Flavobacteria may be major carriers of proteorhodopsin genes in diverse marine environments.

Other genes: The pufM and nasA were not detected in any of the single cell MDA products. However, they were present in six (pufM) and three (nasA) 100-cell MDA reactions (out of a total of twelve), indicating that <1% of bacterioplankton in the sample carried either of these genes. Accordingly, bacteriochlorophyll was previously found to be expressed (infrared fluorescence) in about 1% of bacteria in coastal Maine waters at this time of year (45). All six pufM were 100% identical to each other and were most closely related to bacteriochlorophylls from the Roseobacter lineage (FIG. 2A). Thus, it appears that a single Roseobacter taxon dominated bacteriochlorophyll-containing bacterioplankton in the studied sample. Two nasA were most closely related to assimilatory nitrate reductases in Roseobacter lineage, while one nasA was most closely related to marine gammaproteobacteria (FIG. 2B). The pilot SAG library failed to unambiguously identify these relatively rare but biogeochemically important microorganisms. Screening of a larger SAG library would be an ideal tool for this task. Alternative, community genomics-based analyses have proven less effective to match SSU rRNA and functional genes in such rare taxa.

Genes encoding archaeal SSU rRNA and nitrogenases were not detected in any of the sorted wells, suggesting that Archaea and nitrogen fixing organisms were extremely rare or absent in the analyzed heterotrophic HNA bacterioplankton. Eukaryote SSU rRNA genes were also not detected, confirming effective separation of prokaryotes from protists by FACS.

Conclusions: We demonstrate, for the first time, how a combination of single cell FACS, MDA, and PCR can be used in metabolic mapping of taxonomically diverse, uncultured marine bacterioplankton. Large quantities of high molecular weight whole genome amplification products were obtained from individual cells, allowing for a virtually unlimited number of downstream analyses. In this proof of concept study, we detected proteorhodopsin genes in two out of five flavobacteria, providing evidence that Flavobacteria are major carriers of this photometabolic gene. We also determined that Flavobacteria were a major component of HNA bacterioplankton in the analyzed coastal sample. Fewer than 1% of the analyzed cells carried nasA, pufM, and nifH.

We used standard configuration flow cytometry instrumentation that is available on most major research campuses and is increasingly used aboard oceanographic research vessels. Working at the single cell level requires especially stringent instrument cleaning, sample handling, and quality control methods to prevent DNA contamination. We show that our methods were able to achieve sufficiently low DNA blank controls. The cost of MDA and subsequent PCR-sequencing is in the order of tens of US dollars per cell and thus is significantly less expensive than metagenomic sequencing. In addition to high-throughput screening by PCR or hybridization, SAG libraries may provide material for genomic sequencing of selected, uncultured microorganisms. Two of our SAGs are currently in the process of whole genome sequencing.

### Materials and Methods

Sample collection and single cell sorting. Coastal water sample was collected from Boothbay Harbor, Me., from 1 m depth at the Bigelow Laboratory dock (43°50′39.87″N 69°38′27.49″W) on March 28 at 9:45 AM during high tide (water temperature 7.0° C.). Unmanipulated sample was ten-fold diluted with filtered (0.2 um pore size) sample water and stained with 5 uM (final concentration) SYTO-13 nucleic acid stain (Molecular Probes) for prokaryote detection as in Del Giorgio et al. (46). Individual bacterioplankton were sorted into 96-well plates containing 5 uL per well of phosphate saline buffer (PBS). Only high nucleic acid (HNA) cells were sorted to reduce the probability of depositing dead cells with partially degraded genomes. Single cells were sorted into four of the eight rows on each plate. Of the remaining rows, two were dedicated to background controls, consisting of single drops generated from a sort gate drawn in the “noise” area in the lower left corner of the side scatter/green fluorescence plot. One row of 12 wells was dedicated to blanks with no drop deposition and one row received 100 HNA bacterioplankton cells per well. Sorting was done with a MoFlo™ (Dako-Cytomation) flow cytometer equipped with the CyClone™ robotic arm for sorting into plates, using a 488 nm argon laser and a 70 μm nozzle orifice. The cytometer was triggered on side scatter, the sort gate was based on side scatter and SYTO-13 fluorescence, and “purify 0.5 drop” sort mode was used for maximal sort purity. Extreme care was taken to prevent sample contamination by any non-target DNA. New sheath fluid lines were installed before each sort day. Sheath fluid and sample lines were cleaned by a succession of warm water, 5% bleach solution, and an overnight flush with DNA-free deionized water. Sheath fluid was prepared by dissolving combusted (2 h at 450° C.) NaCl in DNA-free deionized water for a final concentration of 1%. Sorted plates were stored at −80° C. until MDA.

Lysis and multiple displacement amplification (MDA). We compared three protocols for cell lysis, DNA denaturing and MDA in this study:
A. Three cycles of heating to 97° C. and cooling to 8° C. were used for cell lysis and DNA denaturing, after which 18 h MDA was performed using REPLI-g Mini (QIAGEN) Phi29 polymerase and reaction buffer. For each well containing 5 uL PBS, we used 0.5 uL polymerase, 14.5 uL buffer, and 5 uL DNA-free deionized water.
B. Alkaline lysis on ice and 18 h MDA were performed using REPLI-g Mini (QIAGEN) kit reagents and following manufacturer's protocol for blood samples.
C. As Protocol B except that REPLI-g Midi kit (QIAGEN) was used and PicoGreen DNA stain (Invitrogen) was added to the reaction at 0.5x (final concn.). DNA synthesis was monitored with IQ5 real-time PCR system (Bio-Rad). Duplicate standards containing 0.05, 5, 500, and 50,000 fg human genomic DNA (Promega) were amplified simultaneously with the sort samples.

Initially, each of the three protocols were applied on 24 wells: 12 with single cells, 3 no-drop controls, 6 background controls, and 3 with 100 cells. Protocols A, B, and C were employed after 7, 8, and 94 days of storage at −80° C., respectively. Additional 24 wells were analyzed using protocol B after 350 days of storage. The DNA concentration in MDA reactions was determined using a ND-1000 spectrophotometer (Nanodrop) after a cleanup with MinElute PCR Purification Kit (QIAGEN).

PCR-based analyses of MDA products. The MDA products were diluted 10-fold (protocols A and B; REPLI-g Mini kit products) or 200-fold (protocol C; REPLI-g Midi kit products). Two microliters of the dilute products served as templates in 25 uL PCR. Previously described primers and PCR conditions were used to amplify genes encoding bacterial, archaeal and eukaryal SSU rRNA, proteorhodopsin, bacteriochlorophyll, nitrogenase and assimilative nitrate reductase (SI Table 1). The PCR products were cleaned with QIAquick (QIAGEN). For the terminal restriction fragment length polymorphism analyses (T-RFLP) of bacterial SSU rRNA genes, PCR amplicons obtained with 27F-FAM and 907R primers were digested with either HhaI or BsuRI (HaeIII) restriction endonucleases (Fermentas). Sequencing and fragment analyses were performed with 3730x1 analyzer (Applied Biosystems) at the W. M. Keck Center for Comparative and Functional Genomics, University of Illinois at Urbana-Champaign. For T-TRFLP, GeneScan™ 1000 ROX™ Size Standard (Applied Biosystems) was used.

Taxonomic identification of SAGs was achieved by SSU rDNA gene analysis with GenBank BLASTN (47) and Ribosomal Database Project (RDP) Classifier and Seqmatch search tools (48). The SSU rRNA sequences were checked for chimeras using the RDP Chimera Check tool. Protein-encoding sequences were translated with NCBI ORF Finder and their identities verified by GenBank BLASTP searches for closest relatives. Evolutionary trees were constructed using PHYLIP (49) after an automatic sequence alignment with ClustalX (50).

T-RFLP profiling of bacterioplankton communities. Triplicate 1,000 cell aliquots of HNA bacterioplankton were sorted as above into microcentrifuge tubes pre-loaded with 5 uL Lyse-N-Go (Pierce) and then stored at −80° C. Cell lysis was performed according to Lyse-N-Go instructions. Entire lysate volumes were used as templates in 50 uL, 30 cycle PCR reactions using primers 27F and 1492R (Table 2). Two microliter aliquots of these PCR products served as templates in a second, semi-nested, 25 uL and 30 cycle PCR with primers 27F-FAM and 907R. PCR products were cleaned, digested, and fragment analyses were performed as above.

### Quality Control of Single Cell Sorting and Whole Genome Amplification

Multiple displacement amplification. About 0.5 ug and 5 ug of genomic DNA was synthesized in single cell REPLI-g Mini (protocols A and B) and REPLI-g Midi (protocol C) reactions, respectively, with the apparent dominant product size >10 kbp (FIG. 3). Most MDA reactions, including all but two blank treatments, resulted in DNA synthesis. The synthesis of DNA in MDA negative controls has been observed before and is likely caused by Phi29 self-priming, DNA contamination, or both (11, 24, 25). Real-time monitoring of MDA demonstrated that reaction speed depended upon template amount when template was above 5 fg DNA (FIG. 4A). The template-dependent dynamics of MDA observed in this study suggested that 6-8 hours were necessary to complete MDA of fg-level templates using REPLI-g Midi kit (QIAGEN). This agrees with a similar analysis by Zhang et al. (11) but contradicts Spits et al. (18) who suggested 2 h reactions for MDA of single cell genomes.

A log-linear standard curve was produced from the 5 fg, 500 fg, and 50 pg DNA treatments (FIG. 4A insert), and used to calculate tentative DNA concentrations in the sorted samples. The average amount of DNA in the no-drop controls, background controls, single-cell, and 100-cell treatments were estimated to be 0.07 (range 0.0-0.2), 0.78 (0.0-1.8), 2.0 (0.0-10.3) the single and 100 cell treatments was in agreement with published marine bacterioplankton genome size estimates based on fluorometry (2.5 fg) (60), flow cytometry (1.5 fg) (61), and whole genome sequencing data (30, 44). The low DNA content in no-drop controls (mean, 0.07 fg) suggests that sample contamination from handling and reagents was below 4% of an average single cell genome. The background “noise” controls were sorted drops from the low side scatter and fluorescence region. They showed significantly higher DNA (mean, 0.78 fg) than the no drop controls. This DNA could have originated from large viruses, DNA debris, and small (low nucleic acid) bacteria. No DNA was detected in sheath fluid collected at the end of instrument lines. Thus, our sample handling stringency was adequate to prevent significant sample contamination with extraneous genomic DNA.

PCR of the bacterial SSU rRNA gene. Initially, twelve wells with single cells were subjected to each of the lysis-MDA protocols A, B, and C. Using MDA products as templates in PCR, SSU rRNA genes were successfully amplified from 1, 6, and 2 of the wells, respectively (Table 1). Thus, protocol B (cold alkaline lysis and MDA using REPLI-g Mini kit) resulted in the highest success rate (50%) of MDA-PCR. According to manufacturer's statements, we expected similar cell lysis and MDA success rate with REPLI-g Mini and Midi kits, with the Midi kit producing higher DNA yields per sample. The lower success rate of protocol C may be in part explained by possible DNA degradation during prolonged sample storage (94 versus 8 days). After a 350-day storage, the application of protocol B on 12 additional single-cell wells resulted in a 25% success rate. Diverse factors may have contributed to the less than 100% success rate in single cell MDA-PCR, including failed deposition of some cells during FACS, DNA degradation during post-FACS storage, incomplete cell lysis, incomplete MDA, and mismatches to some bacterioplankton groups in the “universal” PCR primers used in this study. It is noteworthy that the 25-50% success rate in MDA-PCR achieved with protocol B was similar to the single cell PCR success rate achieved by Ottesen et al. (10) and to FISH success rate using “universal” SSU rRNA probes (31).

The SSU rRNA PCR products were obtained from all twelve 100-cell wells and from one background control (out of 24 total). No PCR products were obtained from the 12 no-drop controls. High-quality, non-chimerical sequences of near-complete SSU rRNA genes were obtained from all single-cell MDA-PCR products. The SSU rRNA genes of eleven of the twelve SAGs were most closely related to marine isolates and clones (Table 1). One exception, MS024-2C, was identified as Delftia acidovorans, characteristic for soils, freshwaters, and anthropogenic environments. An identical sequence was also retrieved from one of the background controls. This suggests that the sequence is likely a contaminant originating from handling or the reagents. Thus, the overall rate of apparent contamination with bacterial SSU rRNA gene was two out of 84 wells (2%) of single cells, background controls, and no-drop controls combined.

For additional quality control, T-RFLP profiles were generated for the SSU rRNA gene of each PCR-positive single cell and control MDA reaction, using two alternative restriction endonucleases. All profiles contained single peaks (Table 1, FIG. 5A), further confirming that only one cell type was deposited and amplified in each well with no apparent contamination with DNA from other bacterial taxa. It is possible that in some cases aggregates of genetically identical cells, which were optically similar to single cells (e.g. partially divided cells), were sorted into the same well. However, this would not have any adverse effect on the downstream molecular analyses or interpretation.

The HhaI-based T-RFLP profiles generated from 100-cell MDA reactions were dominated by a peak at 55 bp and a group of peaks at around 90 bp (FIG. 5B). Similar T-RFLP profiles were also obtained from 1,000 cell aliquots lysed with Lyse-N-Go protocol and amplified using semi-nested PCR (FIG. 5C). This suggests that the diverse cell lysis and DNA amplification methods used in this study were targeting the same bacterioplankton taxa. The T-RFs found in 100 and 1,000-cell profiles corresponded to those found in SAG profiles of Alphaproteobacteria (55 bp) and Bacteroidetes (around 90 bp; FIG. 5A). The 203 bp Delftia fragment was absent in community profiles, further suggesting its origin as a sample handling contaminant. Conspicuously, the 575 bp T-RF, characteristic to the marine gammaproteobacterium MS024-3A, was also absent in community profiles, either due to PCR biases or rarity of the taxon. On the other hand, 92 bp and 864 bp peaks, present in all 100- and 1000-cell community profiles, were not represented in the SAG library. Considering the relatively small size of the pilot SAG library and the fact that all but two (MS024-1C and MS190-2A) SAG SSU rRNA gene sequences were unique, it is clear that this library covered only a fraction of biodiversity in the bacterioplankton community.

### REFERENCES

1. Pace, N. R., Stahl, D. A., Lane, D. J. and Olsen, G. J. (1986) Advances in Microbial Ecology 9, 1-55.

2. Beja, O., Aravind, L., Koonin, E. V., Suzuki, M. T., Hadd, A., Nguyen, L. P., Jovanovich, S., Gates, C. M., Feldman, R. A., Spudich, J. L., Spudich, E. N. and DeLong, E. F. (2000) Science 289, 1902-1906.

3. Rondon, M. R., August, P. R., Bettermann, A. D., Brady, S. F., Grossman, T. H., Liles, M. R., Loiacono, K. A., Lynch, B. A., MacNeil, I. A., Minor, C., Tiong, C. L., Gilman, M., Osbume, M. S., Clardy, J., Handelsman, J. and Goodman, R. M. (2000) Applied and Environmental Microbiology 66, 2541-2547.

4. Schloss, P. D. and Handelsman, J. (2003) Current Opinion in Biotechnology 14, 303-310.

5. Venter, J. C., Remington, K., Heidelberg, J. F., Halpern, A. L., Rusch, D., Eisen, J. A., Wu, D. Y., Paulsen, I., Nelson, K. E., Nelson, W., Fouts, D. E., Levy, S., Knap, A. H., Lomas, M. W., Nealson, K., White, O., Peterson, J., Hoffman, J., Parsons, R., Baden-Tillson, H., Pfannkoch, C., Rogers, Y. H. and Smith, H. O. (2004) Science 304, 66-74.

6. Tringe, S. G., von Mering, C., Kobayashi, A., Salamov, A. A., Chen, K., Chang, H. W., Podar, M., Short, J. M., Mathur, E. J., Detter, J. C., Bork, P., Hugenholtz, P. and Rubin, E. M. (2005) Science 308, 554-557.

7. DeLong, E. F. and Karl, D. M. (2005) Nature 437, 336-342.

8. DeLong, E. F., Preston, C. M., Mincer, T., Rich, V., Hallam, S. J., Frigaard, N.-U., Martinez, A., Sullivan, M. B., Edwards, R., Brito, B. R., Chisholm, S. W. and Karl, D. M. (2006) Science 311, 496-503.

9. Rusch, D. B., Halpern, A. L., Sutton, G., Heidelberg, K. B., Williamson, S., Yooseph, S., Wu, D., Eisen, J. A., Hoffman, J. M., Remington, K., Beeson, K., Tran, B., Smith, H., Baden-Tillson, H., Stewart, C., Thorpe, J., Freeman, J., Andrews-Pfannkoch, C., Venter, J. E., Li, K., Kravitz, S., Heidelberg, J. F., Utterback, T., Rogers, Y.-H., Falc, n, L. I., Souza, V., Bonilla-Rosso, G., Eguiarte, L. E., Karl, D. M., Sathyendranath, S., Platt, T., Bermingham, E., Gallardo, V., Tamayo-Castillo, G., Ferrari, M. R., Strausberg, R. L., Nealson, K., Friedman, R., Frazier, M. and Venter, J. C. (2007) PLoS Biology 5, e77.

10. Ottesen, E. A., Hong, J. W., Quake, S. R. and Leadbetter, J. R. (2006) Science 314, 1464-1467.

11. Zhang, K., Martiny, A. C., Reppas, N. B., Barry, K. W., Malek, J., Chisholm, S. W. and Church, G. M. (2006) Nature Biotechnology 24, 680-686.

12. Frohlich, J. and Konig, H. (1999) Systematic and Applied Microbiology 22, 249-257.

13. Sieracki, M., Poulton, N. and Crosbie, N. (2005) in Algal Culturing Techniques, ed. Andersen, R. (Elsevier Academic, N.Y.), pp. 101-116.

14. Karl, D. M. and Bailiff, M. D. (1989) Limnology and Oceanography 34, 543-558.

15. Brum, J. R. (2005) Aquatic Microbial Ecology 41, 103-113.

16. Li, H. H., Gyllensten, U. B., Cui, X. F., Saiki, R. K., Erlich, H. A. and Arnheim, N. (1988) Nature 335, 414-417.

17. Pinard, R., de Winter, A., Sarkis, G. J., Gerstein, M. B., Tartaro, K. R., Plant, R. N., Egholm, M., Rothberg, J. M. and Leamon, J. H. (2006) Bmc Genomics 7.

18. Spits, C., Le Caignec, C., De Rycke, M., Van Haute, L., Van Steirteghem, A., Liebaers, I. and Sermon, K. (2006) Human Mutation 27,496-503.

19. Dean, F. B., Hosono, S., Fang, L. H., Wu, X. H., Faruqi, A. F., Bray-Ward, P., Sun, Z. Y., Zong, Q. L., Du, Y. F., Du, J., Driscoll, M., Song, W. M., Kingsmore, S. F., Egholm, M. and Lasken, R. S. (2002) Proceedings of the National Academy of Sciences of the United States of America 99, 5261-5266.

20. Hutchison, C. A., Smith, H. O., Pfannkoch, C. and Venter, J. C. (2005) Proceedings of the National Academy of Sciences of the United States of America 102, 17332-17336.

21. Abulencia, C. B., Wyborski, D. L., Garcia, J. A., Podar, M., Chen, W., Chang, S. H., Chang, H. W., Watson, D., Brodie, E. L., Hazen, T. C. and Keller, M. (2006) Applied and Environmental Microbiology 72, 3291-3301.

22. Hellani, A., Coskun, S., Benkhalifa, M., Tbakhi, A., Sakati, N., Al-Odaib, A. and Ozand, P. (2004) Molecular Human Reproduction 10, 847-852.

23. Jiang, Z. W., Zhang, X. Q., Deka, R. and Jin, L. (2005) Nucleic Acids Research 33.

24. Raghunathan, A., Ferguson, H. R., Bomarth, C. J., Song, W. M., Driscoll, M. and Lasken, R. S. (2005) Applied and Environmental Microbiology 71, 3342-3347.

25. Hutchison, C. A. and Venter, J. C. (2006) Nature Biotechnology 24, 657-658.

26. Kolber, Z. S., Plumley, F. G., Lang, A. S., Beatty, J. T., Blankenship, R. E., VanDover, C. L., Vetriani, C., Koblizek, M., Rathgeber, C. and Falkowski, P. G. (2001) Science 292, 2492-2495.

27. Zani, S., Mellon, M. T., Collier, J. L. and Zehr, J. P. (2000) Applied and Environmental Microbiology 66, 3119-3124.

28. Allen, A. E., Booth, M. G., Frischer, M. E., Verity, P. G., Zehr, J. P. and Zani, S. (2001) Applied and Environmental Microbiology 67, 5343-5348.

29. Buchan, A., Gonzalez, J. M. and Moran, M. A. (2005) Applied and Environmental Microbiology 71, 5665-5677.

30. Moran, M. A., Buchan, A., Gonzalez, J. M., Heidelberg, J. F., Whitman, W. B., Kiene, R. P., Henriksen, J. R., King, G. M., Belas, R., Fuqua, C., Brinkac, L., Lewis, M., Johri, S., Weaver, B., Pai, G., Eisen, J. A., Rahe, E., Sheldon, W. M., Ye, W. Y., Miller, T. R., Carlton, J., Rasko, D. A., Paulsen, I. T., Ren, Q. H., Daugherty, S. C., Deboy, R. T., Dodson, R. J., Durkin, A. S., Madupu, R., Nelson, W. C., Sullivan, S. A., Rosovitz, M. J., Haft, D. H., Selengut, J. and Ward, N. (2004) Nature 432, 910-913.

31. Kirchman, D. L. (2002) FEMS Microbiology Ecology 39, 91-100.

32. Mary, I., Heywood, J. L., Fuchs, B. M., Amann, R., Tarran, G. A., Burkill, P. H. and Zubkov, M. V. (2006) Aquatic Microbial Ecology 45, 107-113.

33. Giovannoni, S. J. and Rappe, M. S. (2000) in Microbial Ecology in the Oceans, ed. Kirchman, D. L. (Wiley-Liss, New York), pp. 47-84.

34. Mary, I., Cummings, D. G., Biegala, I. C., Burkill, P. H., Archer, S. D. and Zubkov, M. V. (2006) Aquatic Microbial Ecology 42, 119-126.

35. Jaspers, E., Nauhaus, K., Cypionka, H. and Overmann, J. (2001) FEMS Microbiology Ecology 36, 153-164.

36. Abell, G. C. J. and Bowman, J. P. (2005) FEMS Microbiology Ecology 51, 265-277.

37. Sabehi, G., Loy, A., Jung, K. H., Partha, R., Spudich, J. L., Isaacson, T., Hirschberg, J., Wagner, M. and Beja, O. (2005) Plos Biology 3, 1409-1417.

38. Gomez-Consarnau, L., Gonzalez, J. M., Coll-Llado, M., Gourdon, P., Pacher, T., Neutze, R., Pedros-Alio, C. and Pinhassi, J. (2007) Nature 445, 210-213.

39. Sabehi, G., Beja, O., Suzuki, M. T., Preston, C. M. and DeLong, E. F. (2004) Environmental Microbiology 6, 903-910.

40. Frigaard, N. U., Martinez, A., Mincer, T. J. and DeLong, E. F. (2006) Nature 439, 847-850.

41. Beja, O., Spudich, E. N., Spudich, J. L., Leclerc, M. and DeLong, E. F. (2001) Nature 411, 786-789.

42. Man, D. L., Wang, W. W., Sabehi, G., Aravind, L., Post, A. F., Massana, R., Spudich, E. N., Spudich, J. L. and Beja, O. (2003) EMBO Journal 22, 1725-1731.

43. Sabehi, G., Massana, R., Bielawski, J. P., Rosenberg, M., Delong, E. F. and Beja, O. (2003) Environmental Microbiology 5, 842-849.

44. Giovannoni, S. J., Tripp, H. J., Givan, S., Podar, M., Vergin, K. L., Baptista, D., Bibbs, L., Eads, J., Richardson, T. H., Noordewier, M., Rappe, M. S., Short, J. M., Carrington, J. C. and Mathur, E. J. (2005) Science 309, 1242-1245.

45. Sieracki, M. E., Gilg, I. C., Thier, E. C., Poulton, N. J. and Goericke, R. (2006) Limnology and Oceanography 51, 38-46.

46. delGiorgio, P., Bird, D. F., Prairie, Y. T. and Planas, D. (1996) Limnology and Oceanography 41, 783-789.

47. Altschul, S. F., Madden, T. L., Schaffer, A. A., Zhang, J. H., Zhang, Z., Miller, W. and Lipman, D. J. (1997) Nucleic Acids Research 25, 3389-3402.

48. Cole, J. R., Chai, B., Marsh, T. L., Farris, R. J., Wang, Q., Kulam, S. A., Chandra, S., McGarrell, D. M., Schmidt, T. M., Garrity, G. M. and Tiedje, J. M. (2003) Nucleic Acids Research 31, 442-443.

49. Felsenstein, J. (1989) Cladistics 5, 164-166.

50. Chenna, R., Sugawara, H., Koike, T., Lopez, R., Gibson, T. J., Higgins, D. G. and Thompson, J. D. (2003) Nucleic Acids Research 31, 3497-3500.

51. Lane, D. J. (1991) in Nucleic Acid Techniques in Bacterial Systematics., eds. Stackebrandt, E. and Goodfellow, M. (John Wiley, Chichester, UK).

52. Page, K. A., Connon, S. A. and Giovannoni, S. J. (2004) Applied and Environmental Microbiology 70, 6542-6550.

53. Vetriani, C., Jannasch, H. W., MacGregor, B. J., Stahl, D. A. and Reysenbach, A. L. (1999) Applied and Environmental Microbiology 65, 4375-4384.

54. Wilms, R., Sass, H., Kopke, B., Koster, H., Cypionka, H. and Engelen, B. (2006) Applied and Environmental Microbiology 72, 2756-2764.

55. Zhu, F., Massana, R., Not, F., Marie, D. and Vaulot, D. (2005) FEMS Microbiology Ecology 52, 79-92.

56. Marie, D., Zhu, F., Balague, V., Ras, J. and Vaulot, D. (2006) FEMS Microbiology Ecology 55, 403-415.

57. Schwalbach, M. S. and Fuhrman, J. A. (2005) Limnology and Oceanography 50, 620-628.

58. Church, M. J., Short, C. M., Jenkins, B. D., Karl, D. M. and Zehr, J. P. (2005) Applied and Environmental Microbiology 71, 5362-5370.

59. Allen, A. E., Booth, M. G., Frischer, M. E., Verity, P. G., Zehr, J. P. and Zani, S. (2001) Applied and Environmental Microbiology 67, 5343-5348.

60. Fuhrman, J. A. and Azam, F. (1982) Marine Biology 66, 109-120.

61. Button, D. K. and Robertson, B. R. (2001) Applied and Environmental Microbiology 67, 1636-1645.

62. Stepanauskas, M and Sieracki, M. E. (2007) Proc Natl Acad Sci. 104, 9052-9057.

63. Rand, K. H. and Houck H (1990) Molecular And Cellular Probes 4, 445-450.

64. Klaschik S., Lehmann L. E., Raadts A., Hoeft A., and Stuber F. (2002) Molecular Biotechnology 22, 231-242.

65. Tseng C. P., Cheng J. C., Tseng C. C., Wang C. Y., Chen Y. L., Chiu D. T. Y., Liao H. C., and Chang S. S. (2003) Clinical Chemistry 49, 306-309.

66. Wages J. M., Cai D. C., and Fowler A. K. (1994) Biotechniques 16, 1014-1017

67. Yang S., Lin S., Kelen G. D., Quinn T. C., Dick J. D., Gaydos C. A., and Rothman R. E. (2002) Journal Of Clinical Microbiology 40, 3449-3454.

68. Sarkar G., and Sommer S. (1990) Nature 347, 340-341.

69. Sharma S., Das D, Anand R., Das T., and Kannabiran C. (2002) American Journal Of Ophthalmology 133, 142-144.

70. Hart J., McArthur J. V., and Stepanauskas R. (2003) Single-Cell PCR in the Identification of Environmental Bacterioplankton, Savannah River Ecology Laboratory, Aiken.

71. Santos S. R., and Ochman H. (2004) Environmental Microbiology 6, 754-759.

72. Yutin N., Suzuki M. T., and Beja O. (2005) Applied and Environmental Microbiology 71, 8958-8962.

73. Allen A. E., Booth M. G., Verity P. G., and Frischer M. E. (2005) Aquatic Microbial Ecology 39, 247-255.

74. Francke C., Siezen R. J., and Teusink B., (2005) Trends in Microbiology 13, 550-557.

75. Kanehisa M., et al. (2004) Nucleic Acids Res. 32, D277-D280.

76. Krieger, C. J. et al. (2004) Nucleic Acids Res. 32, D438-D442.

What is claimed is:

1. A method of analyzing multiple regions of DNA in an individual uncultured cell or an individual uncultured viral particle, the method comprising:
(a) obtaining a sample;
(b) sorting the sample by FACS, thereby obtaining an individual uncultured cell or an individual uncultured viral particle;
(c) amplifying the genome of the individual uncultured cell or the individual uncultured viral particle, thereby producing an amplified genome, and;
(d) analyzing multiple regions of DNA within the cell or viral particle.

2. The method of claim 1, wherein the genome of the individual uncultured cell or the individual uncultured viral particle is amplified through whole-genome multiple displacement amplification.

3. The method of claim 1, wherein analyzing at least one region of DNA within the cell or viral particle is performed by combining the amplified genome with primers that are designed to amplify specific regions of DNA in the amplified genome, under conditions that result in hybridization of the primers to at least one specific region of DNA, thereby determining the occurrence of the specific region of DNA in the amplified genome.

4. The method of claim 1, wherein analyzing regions of DNA within the cell or viral particle is performed by genomic sequencing.

5. The method of claim 1, wherein the method for analyzing regions of DNA within the cell or viral particle is a method for analyzing taxonomic or metabolic markers.

6. A method of analyzing multiple regions of DNA in an individual uncultured microbial cell, the method comprising:
(a) obtaining a microbial sample;
(b) sorting the sample by FACS, thereby obtaining an individual uncultured microbial cell;
(c) amplifying the genome of the individual uncultured microbial cell, thereby producing an amplified genome, and;
(d) analyzing multiple regions of DNA within the cell.

7. The method of claim 6, wherein the individual uncultured microbial cell is an aquatic microbial cell.

8. The method of claim 6, wherein the genome of the individual uncultured microbial cell is amplified through whole-genome multiple displacement amplification.

9. The method of claim 6, wherein analyzing at least one region of DNA within the cell is performed by combining the amplified genome of the cell with primers that are designed to amplify specific regions of DNA in the amplified genome, under conditions that result in hybridization of the primers to at least one specific region of DNA, thereby determining the occurrence of the specific region of DNA in the amplified genome.

10. The method of claim 6, wherein analyzing regions of DNA within the cell is performed by genomic sequencing.

11. The method of claim 6, wherein the method for analyzing regions of DNA within the cell is a method for analyzing taxonomic or metabolic markers.

12. A method of analyzing multiple genes in an individual uncultured microbial cell, the method comprising:
(a) obtaining a microbial sample;
(b) sorting the sample by FACS, thereby obtaining an individual uncultured microbial cell;
(c) amplifying the genome of the individual uncultured microbial cell, thereby producing an amplified genome, and;
(d) analyzing genes within the cell.

13. The method of claim 12, wherein the individual uncultured microbial cell is an aquatic microbial cell.

14. The method of claim 12, wherein the genome of the individual uncultured microbial cell is amplified through whole-genome multiple displacement amplification.

15. The method of claim 12, wherein analyzing at least one region of DNA within the cell is performed by combining the amplified genome of the cell with primers that are designed to amplify specific regions of DNA in the amplified genome, under conditions that result in hybridization of the primers to at least one specific region of DNA, thereby determining the occurrence of the specific region of DNA in the amplified genome.

16. The method of claim 12, wherein analyzing regions of DNA within the cell is performed by genomic sequencing.

17. The method of claim 12, wherein the method for analyzing regions of DNA within the cell is a method for analyzing taxonomic or metabolic markers.

18. A method of analyzing at least one region of DNA in an individual uncultured microbial cell, the method comprising:
(a) obtaining a microbial sample;
(b) sorting the sample by FACS, thereby obtaining an individual uncultured microbial cell;
(c) amplifying the genome of the individual uncultured microbial cell, thereby producing an amplified genome, and;
(d) analyzing at least one region of DNA within the cell.

19. The method of claim 18, wherein the individual uncultured microbial cell is an aquatic microbial cell.

20. The method of claim 18, wherein the genome of the individual uncultured microbial cell is amplified through whole-genome multiple displacement amplification.

21. The method of claim 18, wherein analyzing at least one region of DNA within the cell is performed by combining the amplified genome of the cell with primers that are designed to amplify specific regions of DNA in the amplified genome, under conditions that result in hybridization of the primers to at least one specific region of DNA, thereby determining the occurrence of the specific region of DNA in the amplified genome.

22. The method of claim 18, wherein analyzing regions of DNA within the cell is performed by genomic sequencing.

23. The method of claim 18, wherein the method for analyzing regions of DNA within the cell is a method for analyzing taxonomic or metabolic markers.

24. The method of claim 7, wherein the genome of the individual uncultured microbial cell is amplified through whole-genome multiple displacement amplification.

25. The method of claim 7, wherein analyzing at least one region of DNA within the cell is performed by combining the amplified genome of the cell with primers that are designed to amplify specific regions of DNA in the amplified genome, under conditions that result in hybridization of the primers to at least one specific region of DNA, thereby determining the occurrence of the specific region of DNA in the amplified genome.

26. The method of claim 7, wherein analyzing regions of DNA within the cell is performed by genomic sequencing.

27. The method of claim 7, wherein the method for analyzing regions of DNA within the cell is a method for analyzing taxonomic or metabolic markers.

28. A method for metabolic mapping of an individual uncultured microbial cell, the method comprising:
(a) obtaining a microbial sample;
(b) sorting the sample by FACS, thereby obtaining an individual uncultured microbial cell;
(c) amplifying the genome of the individual uncultured microbial cell, thereby producing an amplified genome;
(d) analyzing at least one region of DNA within the cell, and;
(e) associating at least one region of DNA within the cell with metabolic activity.

29. The method of claim 28, wherein the individual uncultured microbial cell is an aquatic microbial cell.

30. A method of creating a library of single amplified genomes (SAGs) from individual uncultured cells, the method comprising:
(a) obtaining a sample of cells;
(b) sorting the sample by FACS, thereby obtaining individual uncultured cells, and;
(c) amplifying the genomes of the individual uncultured cells, thereby producing a collection of single amplified genomes from individual uncultured cells.

31. The method of claim 30, wherein the individual uncultured cells are microbial cells.

32. The method of claim 31 wherein the microbial cells are aquatic microbial cells.

33. The method of claim 30, wherein the method for creating a library of single amplified genomes (SAGs) is a method for identifying metabolic markers.

34. The method of claim 30, wherein the method for the creation of a library of single amplified genomes (SAG) is a method for whole-genome sequencing of SAGs.

35. A single amplified genome (SAG) library produced by the method of claim 30.

36. The single amplified genome (SAG) library of claim 35 wherein the amplified genomes are marine bacterioplankton amplified genomes.

37. A library of single amplified genomes (SAGs), wherein the library comprises a collection of samples, wherein each sample corresponds to the DNA of an amplified genome of an individual uncultured microbial cell.

38. The method of claim 8, wherein analyzing at least one region of DNA within the cell is performed by combining the amplified genome of the cell with primers that are designed to amplify specific regions of DNA in the amplified genome, under conditions that result in hybridization of the primers to at least one specific region of DNA, thereby determining the occurrence of the specific region of DNA in the amplified genome.

39. The method of claim 8, wherein analyzing regions of DNA within the cell is performed by genomic sequencing.

40. The method of claim 9, wherein analyzing regions of DNA within the cell is performed by genomic sequencing.

41. The method of claim 31, wherein the method for creating a library of single amplified genomes (SAGs) is a method for identifying metabolic markers.

42. The method of claim 31, wherein the method for the creation of a library of single amplified genomes (SAG) is a method for whole-genome sequencing of SAGs.

43. The method of claim 32, wherein the method for creating a library of single amplified genomes (SAGs) is a method for identifying metabolic markers.

44. The method of claim 32, wherein the method for the creation of a library of single amplified genomes (SAG) is a method for whole-genome sequencing of SAGs.

45. The method of claim 33, wherein the method for the creation of a library of single amplified genomes (SAG) is a method for whole-genome sequencing of SAGs.

46. A single amplified genome (SAG) library produced by the method of claim 31.

47. A single amplified genome (SAG) library produced by the method of claim 32.

48. A single amplified genome (SAG) library produced by the method of claim 33.

49. A single amplified genome (SAG) library produced by the method of claim 17.

50. A single amplified genome (SAG) library produced by the method of claim 34.
